(19) United States
(12) Patent Application Publication
(10) Pub. No.: US 2008/0227728 A1
(43) Pub. Date: Sep. 18, 2008

(54) Therapeutic Agent for the Treatment of a Condition Associated with Peripheral Vascular Disease

(75) Inventors: Luis Carvajal Martin, Madrid (ES); Francisco Sevilla Tirado, Zaragoza (ES); Yolanda Aznar Antonanzas, Zaragoza (ES)

Correspondence Address: BERENBAUM, WEINSHIENK & EASON, P.C, 370 17TH STREET, SUITE 4800, DENVER, CO, 80202, US

(73) Assignee: LABORATORIOS BELMAC, S.A., San Sebastian de los Reyes (ES)

(21) Appl. No.: 12/092,549

(22) PCT Filed: Nov. 3, 2006
(86) PCT No.: PCT/EP06/68053

§ 371 Date: May 2, 2008

(30) Foreign Application Priority Data
Nov. 3, 2005 (ES) 200502712

(51) Int. Cl. A61K 31/7048; A61P 9/00

(52) U.S. Cl. 514/27

(57) ABSTRACT

The present invention comprises the use of oleuropein in the preventive and/or therapeutic treatment of a condition associated to the peripheral vascular disease, particularly to venous insufficiency.

The present invention relates to a new use of an already known therapeutic agent.

### STATE OF THE ART

Peripheral vascular disease consists in a damage or obstruction of the blood vessels located farthest from the heart, what affects the peripheral blood circulation. The term “peripheral vascular disease” includes a range of different disorders, which can affect both peripheral arteries and peripheral veins.

Arteriosclerosis of the limbs is among the disorders associated to the peripheral vascular disease affecting peripheral arteries. Arteriosclerosis of the limbs is characterised by the narrowing and the hardening of the arteries which conveys blood to legs and feet. Poor circulation and blockcage of blood in the leg arteries cause pain in the legs, occasionally together with irritation and burn, due to tiredness.

When peripheral vascular disease affects peripheral veins, venous insufficiency occurs. Venous insufficiency is a condition wherein blood vessels do not convey blood efficiently from the lower limbs to the heart. Normally, blood flows through the veins assisted by valves which allow blood to flow upwards to the heart. When these valves are damaged, blood infiltrates and pools in the legs and feet. The symptoms are the inflammation of the legs and dull aching, feeling of heaviness or cramping in the limbs.

Chronic venous insufficiency is a prolonged condition of deficient venous circulation. The onset of said condition is caused by the partial obstruction of veins or by infiltrations in venous valves. In the later stages of the disease, changes in the colouring of the skin and even stasis ulcers can appear.

Venous insufficiency can be accompanied by the onset of varicose veins. Varicose veins are dilated and twisted subcutaneous veins which have lost the capability of maintaining the blood flow in a single direction due to valves malfunction.

The use of compression hoses, with or without additional physiotherapy, is the commonest treatment at the earlier stages of venous insufficiency. The effect of the compression is simply mechanical. This treatment do not affects or corrects the related biologic dysfunction. Furthermore, the treatment with compression hoses often lacks acceptance due to cosmetic and general comfort reasons. In some cases of venous insufficiency it is possible to resort to surgery.

There are commercially available phlebotonic drugs, i.e. those which act improving the venous tone or diminishing the capillary hyperpermeability, intended to ease pain associated to venous insufficiency or even to prevent the progress of the disease. At present, several oral phlebotonic substances have been recalled from the market because of their low therapeutic value. On the other hand, topical administration of said substances causes epithelial irritation problems, allergies or hypersensitivity.

Thus, there is the need of effective and well-tolerated therapeutic alternatives for the treatment of conditions related to the peripheral vascular disease affecting the lower limbs.

### SUMMARY OF THE INVENTION

It has been surprisingly found that oleuropein improves peripheral circulation of individuals showing problems derived from the peripheral vascular disease which affects the lower limbs. Particularly, It has been found that oleuropein has a phlebotonic effect over people showing problems related to venous insufficiency.

Oleuropein is an active ingredient located in the olive tree leaves and in green olives. The cardiovascular effects (Circosta, C. et al. Plantes Medicinales et Phytotherapie, 1990, 24(4), 264-77), the anti-inflammatory effects (Visioli F et al. Life sciences, 1998, 62(6), 541-6), the anti-bacterial effects (Bisignano G; et al. Journal of pharmacy and pharmacology, 1999, 51(8), 971-4), the anti-viral effects (WO 96/14064) and anti-oxidant effects (Saija, A. et al. International Journal of Pharmaceutics, 1998, 166(2), 123-133) of oleuropein have been reported in the literature. However, the state of the art do not teaches or suggests anything related to the use of oleuropein for the treatment of conditions related to peripheral vascular disease affecting the lower limbs, particularly, anything related to its use as a phlebotonic for the treatment of venous insufficiency.

Thus, an aspect of the present invention relates to the use of oleuropein for the manufacture of a medicament for the preventive and/or therapeutic treatment of a condition associated with peripheral vascular disease affecting the lower limbs, or of a disorder derived from said condition, in a mammal, including a human. In a preferred embodiment, the condition is venous insufficiency. In a more preferred embodiment, the condition is chronic venous insufficiency.

Due to its phlebotonic effect, oleuropein is also useful for the treatment of disorders derived from the venous insufficiency. Thus, in an embodiment of the invention, oleuropein is used for the prevention of varicose veins. In another embodiment oleuropein is used for the treatment and/or the prevention of the haemorrhoids.

The invention also relates to a method of preventive and/or therapeutic treatment of a mammal, including a human, which is capable of suffering or is suffering from a condition related to peripheral vascular disease, particularly venous insufficiency. This method comprises the administration to said patient of a therapeutic effective amount of oleuropein, together with pharmaceutically accepted diluents or carriers. The invention further relates to a method of preventive and/or therapeutic treatment of a mammal, including a human, which is capable of suffering or is suffering from a disorder derived from venous insufficiency such as varicose veins or haemorrhoids.

The administration of oleuropein to patients suffering venous insufficiency has the following effects:
inhibition of the venous extravasation (edema),
inhibition and/or elimination of the necrosis provoked by problems of venous return,
inhibition of the angiogenesis provoked by faults on venous circulation or by venous arteriosclerosis,
prevention, delay and/or inhibition of the formation of varicose veins, and
prevention and/or inhibition of haemorrhoids.

Unlike other drugs already known for the treatment of the symptoms derived from a condition related to the peripheral vascular disease, oleuropein do not show significant side effects.

In another preferred embodiment of the present invention, the medicament is in the form of a formulation for topical use. Preferably, said formulation is a transdermal formulation. The use of oleuropein in the form of a transdermal formulation allows a faster absorption of the active ingredient through the skin. An advantage of the oleuropein is that, when administered transdermally, little doses are required to get very significant therapeutic effects, visible and measurable since the first day of its administration. The transdermal formulation can be in the form of an oil, an ointment, a cream or a patch.

In a transdermal formulation, oleuropein has shown specially active at concentrations ranging from about a 0.1% w/w and about a 1% w/w approximately. Said formulations are particularly effective when the content of oleuropein ranges from about a 0.1% w/w and about a 0.4% w/w. The percentages are by weight based on the final ingredients of the composition.

The term “phlebotonic” or “phlebotonic pharmaceutical product” relates to substances which act improving the venous tone or diminishing capillary hyperpermeability.

The term “transdermal formulation” relates to a formulation which is absorbed through the skin.

Throughout the description and claims the word “comprise” and its variations is not intended to exclude other technical features, additives, components, or steps. All the percentages are by weight. The content of the application from which priority is claimed, as well as the contents of the abstracts of the priority application and of the present application, are incorporated herein as reference. Additional objects, advantages and features of the invention will become apparent to those skilled in the art upon examination of the description or may be learned by practice of the invention. The following examples and drawings are provided by way of illustration, and are not intended to be limiting of the present invention.

### EXAMPLES

The effects of the treatment with oleuropein in patients suffering from disorders of peripheral circulation had been determined in several patients suffering from venous insufficiency. Several formulations containing oleuropein at different concentrations to be administered were prepared .

### Example 1

### Preparation of an Oil

25 kg of a composition were prepared following the following process:

### a) Preparation of the Aqueous Phase.

62.5 g of oleuropein 40% w/w (a 0.1% w/w of oleuropein), 5,000 g of diethyleneglycol monoethyl ether, and 12.5 g of benzoic acid were added to a stainless steel tank. The mixture was stirred until complete dissolution.

### b) Preparation of the Oily Phase.

The remaining components, i.e. 17,120 g of virgin olive oil, 1,250 g of oleic acid, 1,500 g of 15-pentadecalactone, and 25 g of sorbitan monoestarate (Span 60), were added to a second stainless steel tank. The mixture was heated between 60 and 65° C. under continuous stirring for 30 minutes more, until total homogenization.

### c) Obtaining the Oil

The mixture obtained at step b) was added over the mixture obtained at step a) with stirring. 22.5 g of bergamot oil, and 7.5 g of tea-tree aroma were added as flavours and the mixture was homogenized by stirring until room temperature.

Three additional oil compositions were prepared following the same process containing a 0.2% w/w, a 0.4% w/w and a 0.8% w/w of oleuropein, respectively.

### Example 2

### Preparation of an Ointment

25 kg of a composition were prepared following the process:

### a) Preparation of the Aqueous Phase.

250.0 g of oleuropein 40% w/w (a 0.4% w/w of oleuropein), and 500 g of diethyleneglycol monoethyl ether were added to a stainless stain tank. The mixture was stirred until total dissolution.

### b) Preparation of the Oily Phase

14,470 g of virgin olive oil, 2,500 g of oleic acid, 750 g of 15-pentadecalactone, 6,000 g of cetyl alcohol and 500 g of sorbitan monoestarate (Span 60), were added to a second stainless steel tank. The mixture was heated until approximately 50° C. under a continuous stirring until total homogenization.

c) Obtaining the Ointment

The mixture obtained in step b) was added over the mixture obtained in step a) under stirring. 22.5 g of bergamot oil and 7.5 g of tea-tree aroma were added as flavours and the mixture was homogenized by stirring until it cooled down to room temperature.

Two additional oil compositions respectively containing a 0.1% w/w and a 0.2% w/w of oleuropein, respectively, were prepared following the previous process.

### Example 3

### Preparation of a Cream

25 kg of a composition were prepared following the following process:

### a) Preparation of the Aqueous Phase.

25 g of metilparaben, 12.5 g of propilparaben, 240 g of polyoxiethylene sorbitan monoestearate (Tween-60) and 12.6 L of water were added to a stainless stain tank. The mixture was stirred at about 50° C. until total dissolution.

### b) Preparation of the Oily Phase

6,250 g of virgin olive oil, 1,250 g of oleic acid, 1,500 g of 15-pentadecalactone, 1,875 g of cetyl alcohol, and 260 g of sorbitan monoestarate (Span 60), were added to a second stainless steel tank. The mixture was heated at about 50° C. under a continuous stirring until total homogenization.

### c) Dissolution of the Physiologically Active Agent

125.0 g of oleuropein 40% w/w (a 0.2% w/w of oleuropein) were dispersed over 595 g of diethyleneglycol monoethyl ether at room temperature.

### d) Obtaining the Cream

The mixture obtained in step b) was added over the mixture obtained in step a) under stirring in an Ultra-Turrax type system until the homogenization of the mixture. Next, the mixture obtained in step c) was added by stirring together with

12.3 g of Shine aroma and it was let to cool down until room temperature.

Following the previous process, two additional cream compositions were prepared containing 0.1% w/w and 0.4% w/w of oleuropein, respectively.

Several tests were carried out over some patients suffering from chronic venous insufficiency to prove the effects of oleuropein.

### Example 4

### Effects of a Topical Formulation

A patient suffering from chronic venous insufficiency presented a lesion on the back of the leg due to a devitalization of the dermal tissues, showing a brown coloration of the tissue, characteristic of an incipient necrosis. Between 1 and 2 ml of a topical composition according Example 1, containing oleuropein at a concentration of a 0.2% w/w, were applied every 12 hours over the leg of said patient. The treatment was maintained for 15 days. After this period the treatment was continued lowering the administration to once a day.

After two months of treatment, the brown coloration of the tissue was clearly diminished. A revitalization both of the central area of the lesion, showing sane tissue with normal coloration, and of the borders, wherein brown pigmentation was diminished, was noticed

### Example 5

### Effects of a Topical Formulation

The leg of a patient suffering from chronic venous insufficiency showed the general appearance of a clear congestive type circulatory deficit (bluish colour) with compromised tissue vitality and elevated inflammatory areas. Some zones, extended over the side and the base of the leg, showed erythematosus, inflammatory, congestive lesions and dermal scales.

Between 1 and 2 ml of a topical composition according Example 1 containing oleuropein 0.2% w/w, were administered every 12 hours over the leg of said patient. The treatment was maintained for 15 days. After this period the treatment was continued lowering the dose administration to once a day.

After one month of treatment, the general volume of the limb was clearly diminished. The damaged tissue area also diminished, which was apparent by the general colouring change (from a dark bluish colour to a more pink tone), the diminishing of oozings and dermal scabs, and a significant decline of the elevated inflammatory areas. External signs made apparent an improvement on tissue perfusion, proving the presence of a more lively tissue.

1. (canceled)

2. (canceled)

3. (canceled)

4. (canceled)

5. (canceled)

6. (canceled)

7. (canceled)

8. (canceled)

9. (canceled)

10. A method for one or both the preventive and/or therapeutic treatment of a mammal, including a human, which is capable of suffering or is suffering from venous insufficiency or from a disorder derived from venous insufficiency; the method comprising administering to said mammal a therapeutically effective amount of oleuropein.

11. A method according to claim 10 wherein the administering step includes administering the oleuropein together with one or more pharmaceutically accepted diluents or carriers.

12. The method according to claim 10, wherein the venous insufficiency is chronic venous insufficiency.

13. The method according to claim 10, wherein the disorder is haemorrhoids.

14. The method for the preventive treatment according to claim 10, wherein the disorder is varicose veins.

15. The method according to claim 10, wherein the oleuropein is administered in a formulation for topical use.

16. The method according to claim 15, wherein the formulation for topical use is a transdermal formulation.

17. The method according to claim 16, wherein the formulation comprises an amount of oleuropein between about 0.1% w/w and about 1% w/w.

18. The method according to claim 17, wherein the amount of oleuropein is comprised between about 0.1% w/w and about 0.4% w/w.
